(19) United States
(12) Patent Application Publication
(10) Pub. No.: US 2014/0336463 A1
(43) Pub. Date: Nov. 13, 2014

(54) Rhinotopic therapy for the treatment of chronic rhinosinusitis biofilm disruption, treatment of mucosal granulation and sinus polyps

(71) Applicant: Alan H. Shikani, Ruxton, MD (US)

(72) Inventor: Alan H. Shikani, Ruxton, MD (US)

(21) Appl. No.: 12/657,557

(22) Filed: Jan. 22, 2010

Related U.S. Application Data
(60) Provisional application No. 61/210,142, filed on Mar. 16, 2009.

(51) Int. Cl. A61M 11/00; A61M 3/02; A61M 31/00

(52) U.S. Cl. CPC A61M 11/00; A61M 31/00; A61M 3/0279; A61M 3/0295; A61M 2210/0681
USPC 600/158; 604/514; 604/131; 604/523

(57) ABSTRACT

The herein disclosed invention is directed to methods of treating difficult to treat refractory or recalcitrant rhinosinusitis involving the steps of (1) topical application of antimicrobials/antibiotics and corticosteroids, (2) endoscopic debridement and cleansing to remove debris and biofilm, (3) endoscopic instillation of antimicrobials/antibiotics plus corticosteroids to the sinuses, and (4) hydrotherapy to disrupt/inhibit bacterial/fungal biofilm attachment to the sinus mucosa. Also disclosed is a kit of components for carrying out the treatment of this invention.

### RELATED APPLICATIONS

This application is related to Provisional Application 61/210,142 filed Mar. 16, 2009.

### FIELD OF THE INVENTION

The herein disclosed invention finds applicability in the field of treatment of rhinosinusitis and related medical condition affecting the sinus lining.

### BACKGROUND OF THE INVENTION

Rhinosinusitis is an infection/inflammation of the lining of the membranes of the nose and paranasal sinuses. The sinuses are air-filled cavities in the skull that are lined with ciliated stratified columnar epithelium and are contiguous with the upper respiratory tract via the sinus ostia. Inflammation of the sinuses causes mucosal edema and increased sinonasal secretions and obstruction of the ostium that aerates the sinuses. If sinus obstruction occurs, the retained secretions create a milieu that is ideal for bacterial growth resulting in bacterial sinusitis. While the most common etiology is an upper respiratory tract infection, an acute exacerbation of allergic rhinitis, dental infection or manipulation, or trauma to the sinuses may also be causative.

Chronic rhinosinusitis is a very common disease and affects about 40 million people each year in the U.S. Rhinosinusitis is defined as an inflammation of the sinus lining commonly caused by bacterial, viral and/or microbial infections that cause structural issues such as sinus ostium blockage (the ostium is the window of the sinus). Symptoms include nasal congestion, facial discomfort, nasal discharge, headache, and fatigue. Sinusitis is classified as acute or chronic based on the duration of the infection. Acute sinusitis has been defined by the American Academy of Otolaryngology—Head and Neck Surgery as a sinus infection in which symptoms last less than 4 weeks. If the symptoms persist beyond 12 weeks of treatment, the sinusitis is labeled as chronic.

The most commonly used treatment for chronic sinusitis consists of medications such as antibiotics and/or topical nasal corticosteroids, which are often successful in reducing mucosal swelling and relieving obstruction of the sinus ostium and ostio-meatal region. The ostio-meatal complex (anterior ethmoid-middle meatal region) is a key area in the pathogenesis of sinusitis. It contains the narrow channels that provide for mucociliary clearance and ventilation of the interior ethmoid, maxillary, and frontal sinuses. Relatively minor swelling of the mucosa in this area, such as those associated with viral upper respiratory tract infections or allergic rhinitis, may lead to frontal or maxillary sinus obstruction and secondary disease within these sinuses. Foreign bodies, including nasogastric tubes, nasotracheal tubes, and nasal packing risk blocking the ostio-meatal complex and causing nosocomial sinusitis. When sinus drainage is obstructed, mucus, inflammatory cells and bacteria accumulate, oxygen tension in the sinuses is reduced, and opsonization/phagocytosis as well as immunoglobulin-dependent activities are impaired and pathogenic bacteria multiply causing clinical sinusitis.

Multiple organisms have been isolated from the sinuses of patients with sinusitis. However, it is important to note that “normal flora” has been cultured from healthy uninfected sinuses. “Normal flora” include Streptococcus pneumoniae, Haemophilus influenzae, Staphylococcus aureus, and anaerobes such as Bacteroides species, anaerobic gram-positive cocci, and Fusobacterium species. The presence of these bacteria is only “normal” if the titer of resident flora is low, but when bacterial titers become high they become pathogenic.

The primary bacterial pathogens in acute sinusitis are Streptococcus pneumoniae, Haemophilus influenzae, and Branhamella catarrhalis. Bacteroides species and Staphylococcus aureus are found in 8% to 10% of patients with acute sinusitis. Chronic sinusitis may be usually associated with anaerobes, most commonly Bacteroides species, anaerobic gram-positive cocci, Fusobacterium species, Streptococcus, Veillonella, and Corynebacterium species. Gram-negative bacteria predominate in nosocomial sinusitis, including Pseudomonas aeruginosa, Klebsiella pneumoniae and Enterobacter species.

Fungi are an uncommon cause of sinusitis. Organisms that infect the sinuses only rarely include Aspergillus, Actinomyces species and Nocardia species. There should be a high index of suspicion for fungal sinusitis (e.g., sinusitis induced by mycosis caused by fungi of the genera Aspergillus, Bipolaris, Candida, and Penicillium and of the order Mucorales) in immunocompromised patients and in those that do not respond to antibiotic therapy.

Sinusitis is managed medically with oral antibiotics, nasal corticosteroids, decongestants and by avoidance of any exacerbating environmental factors. Oral decongestants such as pseudoephedrine or phenylpropanolamine are helpful but should be used with care in hypertensive patients. In some instances, oral corticosteroids may offer additional relief, especially in cases with allergies or significant polyposis. A nasal spray (e.g., oxymetazoline) may be added to the therapeutic regimen, but should be used for no more than 3 to 5 days. Traditional approaches to treatment, such as steam or mist inhalation, are also often not prescribed, but may relieve both the discomfort and drainage associated with sinusitis. Patients with signs and symptoms suggestive of allergic sinusitis, and patients with an exacerbation of chronic sinusitis, are also treated with anti-inflammatory corticosteroid nasal sprays such as fluticasone (Flonase), beclomethasone (Beconase, Vancenase), triamcinolone (Nasacort), mometasone (Nasonex) budenoside (Rhinocort) or flunisolide (Nasarel). Prolonged use of topical steroids and short bursts of oral steroid therapy may help to reduce swelling and relieve osteomeatal obstruction. Allergic patients also benefit from the use of antihistamines and/or immunotherapy or desensitization.

A broad-spectrum antibiotic is typically chosen to cover the usual sinus pathogens (H influenzae, S pneumoniae, and B catarrhalis). Amoxicillin, may be used as an initial treatment for management of sinusitis, but there is an increasing incidence of resistance to penicillins. Cephalosporins provide more powerful alternatives for nonresponders. The new cephalosporins include cefpodoxime (Vantin), ceftibuten (Cedax), cefuroxime, loracarbef (Lorabid), or cefaclor (Ceclor PD). Another effective alternative is amoxicillin/clavulanate potassium (Augmentin). Augmentin is the only antibiotic that is approved for chronic sinusitis. Clarithromycin (Biaxin) may be given to patients who are allergic to penicillins. Acute sinusitis frequently manifests with heavy bacterial growth of a predominant pathogen, but chronic sinusitis is typically a polymicrobial infection in which anaerobes are often present. Therefore, broad-spectrum coverage should be provided, and prolonged therapy (up to several weeks) may be required.

If medical treatment fails to relieve the symptoms of rhinosinusitis, a repeat course, implementing a different, possibly broader-spectrum antibiotic is prescribed. If that fails, then the patient is considered a good candidate for endoscopic sinus surgery (or for balloon sinuplasty). The principle behind surgery is that removing the obstruction in the anterior ethmoids will permit spontaneous resolution of disease in the ethmoids, maxillary and frontal areas.

Many studies have been published evaluating endoscopic sinus surgery (ESS) for the treatment of chronic rhinosinusitis (CRS), and little doubt exists as to its efficacy in the hands of experienced surgeons. Significant improvement in symptoms (97 to 98%) has been reported in the first 12 months post-operatively (1-3), but this does not seem to be long-lasting. Three year-outcome analysis shows that the beneficial effect dissipates over time, and a gradual worsening of the symptom score has been noted in most major symptoms including nasal congestion, anterior & posterior nasal discharge, and anosmia/hyposmia (4-6). Some rhinologists even consider ESS a failed long-term treatment for the complaint of posterior nasal discharge (4). Persistent symptom relief after ESS has been reported to vary between 75 and 95% of the patients, but 5 to 25% continues to be symptomatic despite rigorous medical management. These patients are treated with different and sustained regimens of oral antibiotics and nasal steroids, and sometimes with oral steroids or even intravenous antibiotics, but not all patients respond, in fact many continue with chronic symptoms or recurrent acute flares over time (8, 9).

The management of patients who continue to experience sinus symptoms after ESS (referred to in the literature as “refractory” or “recalcitrant” sinusitis) is complex and challenging. To start, it is difficult to determine a single underlying etiology for these recurrent infections. This is due to the fact that the basis of this disease is most likely multifactorial and that several issues need to be addressed at the same time in order to achieve success. First, one needs to correctly identify the pathogenic organism(s) and make sure that we are not just dealing with colonizing bacteria before choosing the optimal antibiotic. This can be made difficult by the presence of biofilms, which are often present in CRS and which have the capacity to trap and shield bacteria (21-29). A biofilm is an aggregate of cells stuck to each other and/or to a surface and which are frequently embedded within a self-produced matrix of extracellular polymeric substance (EPS), also referred to as “slime”. In the infected sinuses, unless the biofilm is shedding sufficient planktonic bacteria that can be cultured, it is possible to miss the real pathogen. Indeed there is evidence in the literature that the bacteria inside the biofilms and swab cultures do not always correlate (28, 29). This EPS encasement provides bacteria with a method of evading the mucociliary clearance mechanism of the sinuses, and also resists conventional oral antibiotics therapies. Biofilms are usually bacterial but in some instances, both bacteria and fungi coexist in biofilms of patients with CRS (29). Surface adhesion is an efficient means of lingering in a favorable environment rather than being swept away by the current. Once bacteria are freed from their plastic encasement, they are more likely to be exposed to and killed by antibiotics. Effective biofilms treatment necessitates the following: disruption of the biofilm three-dimensional structure, increasing antibiotics penetration and bacterial susceptibility, and interrupting the bacterial quorum sensing signals by which biofilms bacteria interact with neighboring cells (28, 44, and 45). Reaching antibiotic concentrations to levels above the MIC (Minimal Inhibitory Capacity) levels inside the biofilm is difficult with oral antibiotics alone, and it is easier to achieve using a topically applied antibiotic (46). Another advantage of topical delivery is the minimal systemic side effects, due to little drug absorption.

Controlling infection at the mucosal level is a key factor for successful treatment of refractory CRS, however controlling the mucosal inflammatory process is equally important as there is an intricate association between infection and inflammation. On one hand, bacterial biofilms create and perpetuate the inflammatory reaction that underlies refractory sinusitis, and on the other hand, inflammation damages the epithelium, impairs local defenses and subsequently favors bacterial attachment with biofilms formation (22, 23 and 25). This is why, in addition to eradicating infection, controlling the inflammatory process is key to a successful treatment. The inflammatory process includes increased capillary permeability, leukocytes infiltration, angiogenesis and edema, which ultimately leads to granulation tissue. Corticosteroids exercise multiple immunomodular functions including stabilization of mast cells against mediator release, blocking the formation of inflammatory mediators, and inhibiting chemotaxis of inflammatory cells. Corticosteroids also inhibit angiogenesis and granulation tissue formation (40). Finally, corticosteroids inhibit the allergic hypersensitivity reaction, which may play a significant effect in the pathogenesis of rhinosinusitis. Corticosteroids can be administered orally, however topical administration is associated with less side-effects. The experience of the otologists has shown that the addition of topical corticosteroids to ototopical antibiotic preparations is quite effective in reducing granulation, and provides an important clinical advantage over antibiotics alone when treating otitis externa or otitis media with otorrhea (40-43).

If patients with refractory rhinosinusitis continue to be symptomatic despite standard extended medical therapy, the physician should consider revision surgery. Surgery is usually indicated in the presence of ostial obstruction, scarring or residual disease, that is confirmed by CAT scan. In our experience, many patients with refractory CRS have patent antrostomies and are not necessarily obstructed; however they do show evidence of inflamed and even infected polypoid mucosa. These patients may be candidates for surgery but with the realization that revision surgery may carry higher surgical risks of major complications than the original procedure (30-34). Another drawback of surgery is that at best, it is associated with varying degrees of success; in fact a past history of unsuccessful ESS is more likely to be associated with failure of revision surgery (35). It is therefore evident that there is a need to develop newer medical alternatives to treat refractory rhinosinusitis.

The scope of this invention (which we term “rhinotopic therapy”) is to provide a new medical treatment alternative, which raises the threshold for the need of revision surgery, or allows the patient to avoid surgery all together.

### DESCRIPTION OF RELATED ART

Osbakken et al (U.S. Pat. No. 7,128,897) is directed to aerosolized anti-infectives, anti-inflammatories, and decongestants for the treatment of sinusitis. Pharmaceutical compositions are described that contain one agent for treatment of sinusitis and a surfactant. The compositions are prepared to have a surface tension that renders the composition effective for treatment of sinusitis. The herein disclosed invention is for a method patentably distinct from that of Osbakken.

### BRIEF SUMMARY OF THE INVENTION

The invention is directed to a novel method and algorithm for the treatment of chronic and/or refractory rhinosinusitis. The inventor calls his method rhinotopic therapy. Rhinotopic therapy is a method of treatment which combines four steps:
Step 1: Regular topical delivery of antibiotics/antimicrobials and corticosteroids to the infected/inflamed sinuses using a nebulizer.
Step 2: Endoscopic debridement and cleansing of the sinus cavities from debris and from biofilms that adhere to the sinus mucosa.
Step 3: Endoscopic instillation (by spray, gel/ointment, polymer or lipospere) of antibiotics/antimicrobials and corticosteroids directly inside the sinus cavities or the areas around the sinuses, such as the middle meatus or the inferior meatus.
Step 4: Regular hydrotherapy with saline douches or irrigation, (with or without chelating agents or surfactants or mucolytics) that disrupts biofilms and inhibits bacterial/fungal attachment to the sinus mucosa.
   These steps will be described in greater detail below.

### DEFINITIONS

Antimicrobials are defined to include antibiotics, antifungals, and/or antivirals, and are described and listed in the text below.

Chelating agents, surfactants and mucolytics are described and listed in the text below.

CRS=chronic rhinosinusitis.

ESS=endoscopic sinus surgery.

Rhinotopic Rx=Rhinotopic Therapy.

### OBJECTS OF THE INVENTION

A main object of the invention is to produce a method for treating cases of chronic rhinosinusitis and/or refractory rhinosinusitis.

A further object of the invention is to treat complex sinus diseases for which standard medical or surgical therapy is not effective or too risky.

An object of the invention is to use a multifaceted comprehensive approach to treat refractory or recalcitrant rhinosinusitis.

Yet another object of the invention is to carry out rhinotopic therapy as complementary to endoscopic sinus surgery for chronic rhinosinusitis.

Yet another object of the invention is to carry out rhinotopic therapy as a substitute for endoscopic sinus surgery for chronic rhinosinusitis.

These and other objects of the present invention will become apparent from a reading of the following specification in conjunction with the enclosed drawings.

### BRIEF DESCRIPTION OF THE DRAWINGS

FIG. 1 is a chart showing Lund-Kennedy scores of refractory chronic rhinosinusitis patients receiving spray Rhinotopic Therapy.

FIG. 2 is a line graph showing Lund-Kennedy Scores of refractory chronic rhinosinusitis patients receiving spray Rhinotopic Therapy.

FIG. 3 is a line graph showing the Percentage Improvement in Lund-Kennedy Scores in Response of refractory chronic rhinosinusitis patients receiving spray Rhinotopic Therapy.

FIG. 4 is a column graph showing the response of refractory chronic rhinosinusitis patients to spray Rhinotopic Therapy.

FIG. 5 is a line graph showing Lund-Kennedy Scores of refractory chronic rhinosinusitis patients receiving gel Rhinotopic Therapy.

FIG. 6 is a line graph showing the Percentage Improvement in Lund-Kennedy Scores in Response of refractory chronic rhinosinusitis patients receiving gel Rhinotopic Therapy.

FIGS. 7 and 8 are line graphs showing the percentage improvement and the Lund Kennedy scores of MRSA (Methicillin Resistant Staph Aureus) refractory sinusitis patients to Rhinotopic Therapy.

FIGS. 9 and 10 are a column graph and a line graph showing the response of Sarcoidosis patients to Rhinotopic Therapy.

FIG. 11 is a column graph showing the response of Samter's patients to Rhinotopic Therapy.

FIG. 12 describes a kit of components for carrying out Rhinotopic Therapy.

FIG. 13 is an algorithm for the “Rhinotopic Therapy” method of treating refractory CRS.

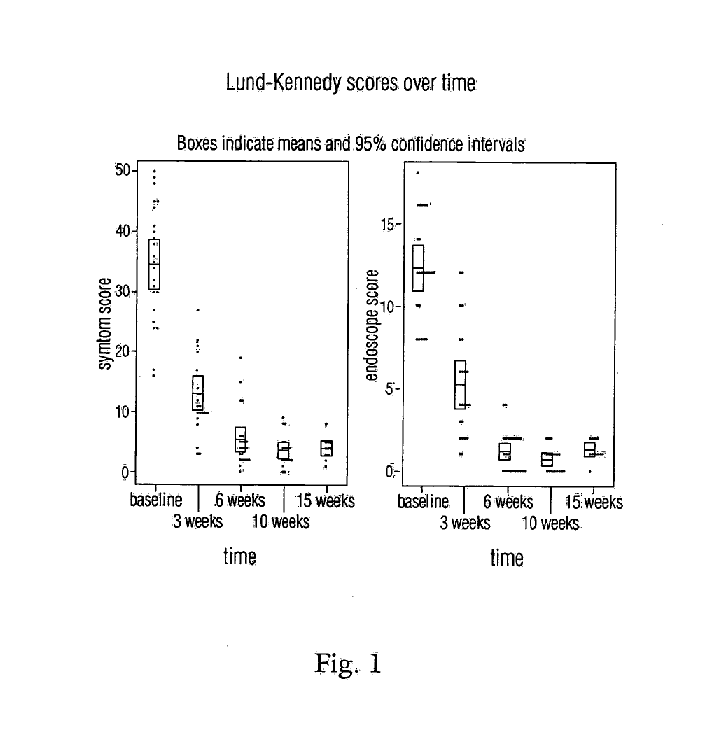

### DETAILED DESCRIPTION OF THE INVENTION

A novel method and algorithm for the treatment of chronic and/or refractory rhinosinusitis is presented. The inventor is introducing this method for the first time and is calling it “Rhinotopic Therapy”. Rhinotopic therapy is a method of treatment that combines four steps: Step 1. Regular topical delivery of antibiotics/antimicrobials and corticosteroids to the infected/inflamed sinuses using a nebulizer. This is done through a small-particle or large particle nebulizer that aerosolizes antibiotic for the treatment of infection, and corticosteroids for the treatment of inflammation and granulation. Step 2. Endoscopic debridement and cleansing of the sinus cavities from debris and from and biofilms that likely adhere to the sinus mucosa. Step 3. Endoscopic instillation of antibiotics/antimicrobials+corticosteroids directly inside the sinus cavities or the areas around the sinuses, such as the middle meatus or the inferior meatus. This part of the process can be done either by spraying with antibiotics/antimicrobials+corticosteroids through a topical atomizing device, or by applying a gel and/or ointment, polymer or liposphere that contains a depot of antimicrobials+corticosteroids and releases them locally as it degrades. Gels and/or ointments have the capacity for a sustained topical drug delivery over a period of time. Lipospheres are small lipid spherical particles with an internal hydrophobic core composed of fats and biodegradable polymers (mainly triglycerides and lactide-based polymers) whereas the surface is composed of, for example, lecithin/phospholiped molecules, and that have the capacity for a sustained topical drug delivery for an even more extended period of time. Step 4. Regular hydrotherapy with saline douches or irrigation (with or without the addition of chelating agents, surfactants or mucolytics that inhibit bacterial/fungal attachment to the sinus mucosa).

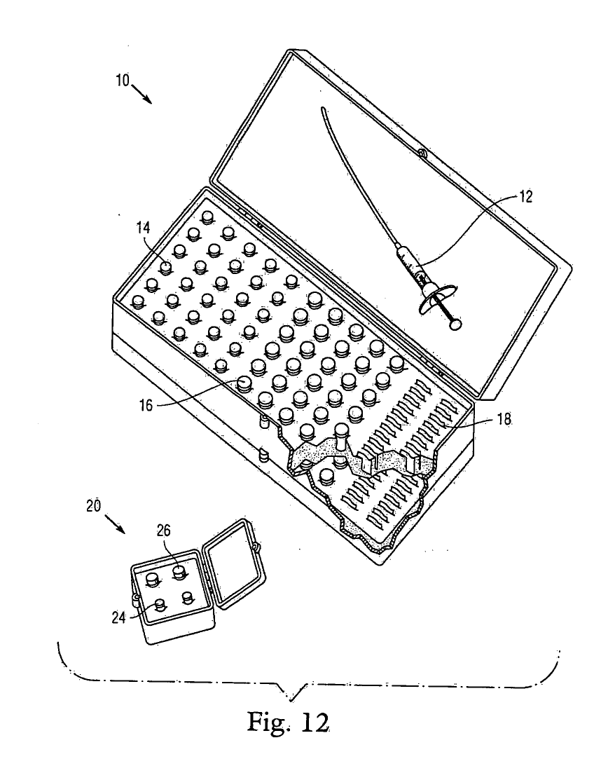

A special feature of this invention is a kit containing components necessary for performing Rhinotopic therapy. For convenience and accuracy of dosage, the medicaments and instruments used for treatment are supplied to the doctor or the patient in a kit. With reference to FIG. 12 a kit 10 to be used by the patient shows: 1) A supply of antibiotic 14 and a supply of corticosteroids 16 which medications can be given throughout the duration of rhinotopic therapy. The medications can be provided as liquid/gel/ointment and/or lipospheres that are pre-loaded in syringes, ampoules or other small medicine containers. 2) A kit 20 (see FIG. 12) for the physician to deliver the medications to the sinuses after debridement under guidance of an endoscope. The kit 10 has a mucosal atomizer device 12 that is used to atomize/spray the liquid medication or instead the kit could contain an injection cannula that is used to deliver the gel/ointment, or by liposheres that contain the medications. 3) Is a supply of pre-packaged 18 sodium chloride powder and/or sodium bicarbonate powder that can be diluted with water to reconstitute the saline that will be used in hydrotherapy. Additional components such as chelating agents, surfactants or mucolytics can be added to the package 18. 4) The kit can alternatively also include a nebulizer device to be used by the patient to nebulize the medications to the sinuses. Note also that the kit 10 has two layers of medications as is shown by the kit broken away at lower right in FIG. 12.

The inventor has demonstrated in this invention that Rhinotopic Therapy is a highly effective treatment modality that successfully restores mucosal health and sinus homeostasis in the majority (90%±) of cases of refractory chronic rhinosinusitis, an entity for which standard medical therapy (which usually includes repeated oral antibiotics and commercial nasal corticosteroids) usually does not work. The inventor has also shown Rhinotopic Therapy to be a well tolerated treatment modality, that safely allows topical administration of antibiotics that can otherwise only be administered intravenously with significant potential side-effects.

For the cases where the rhinotopic treatment does not work from the first time, an algorithm is presented whereby a repeat rhinotopic treatment is provided. If the symptoms persist, then the patient's options may include a long term/sustained rhinotopic therapy, or alternatively intravenous antibiotics or revision sinus surgery.

In greater detail, the invention presents a novel method and algorithm for the disruption of biofilm and control of the inflammatory reaction and or polyps of the sinus mucosa using Rhinotopic Therapy (with or without chelating agents). As previously described, Rhinotopic Therapy consists of a combination of small-particle or large-particle nebulized antibiotics/antimicrobials for the treatment of bacterial biofilms along with small-particle or large-particle nebulized corticosteroids for the treatment of granulation, and with biofilm debridement and cleansing, using endoscopic instillation of antibiotics/antimicrobials and corticosteroids directly inside the sinus cavities and with hydrotherapy with saline douches or irrigation, with or without surfactants and mucolytics to break down bacterial/fungal biofilms and to further inhibit their attachment to the sinus mucosa.

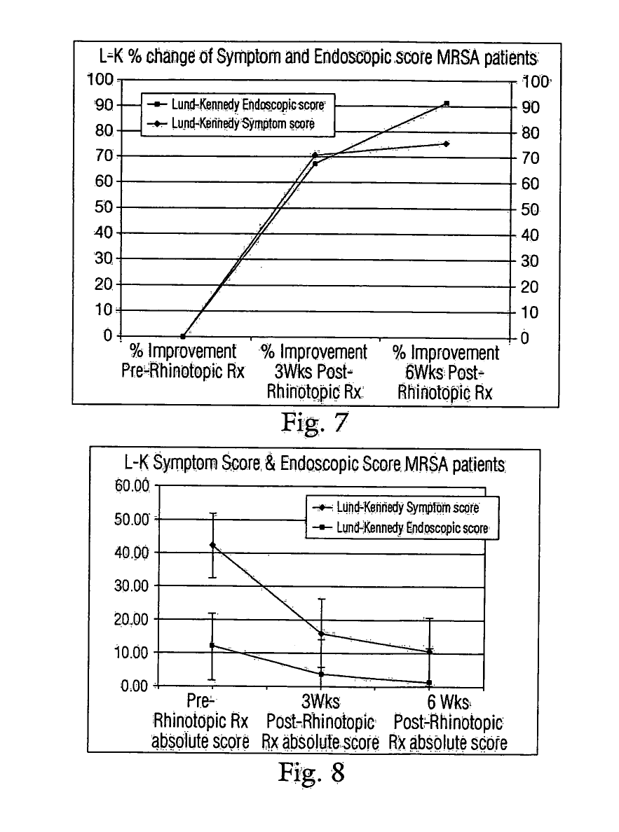

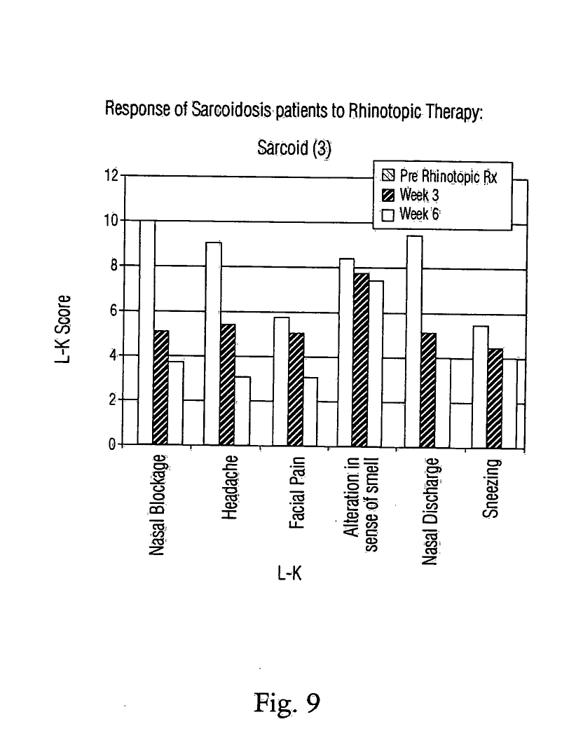

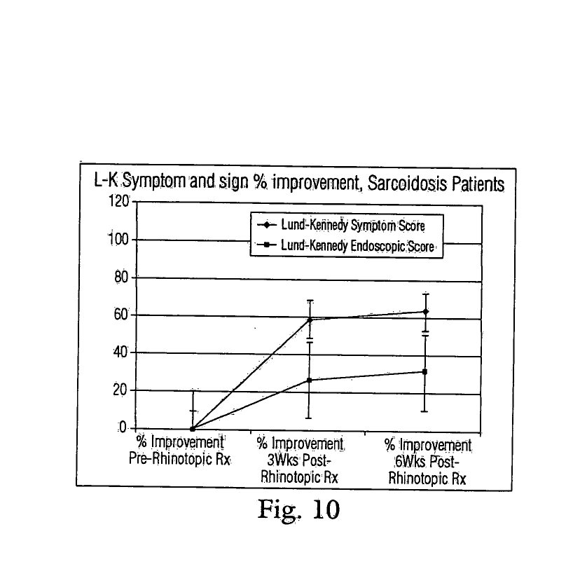

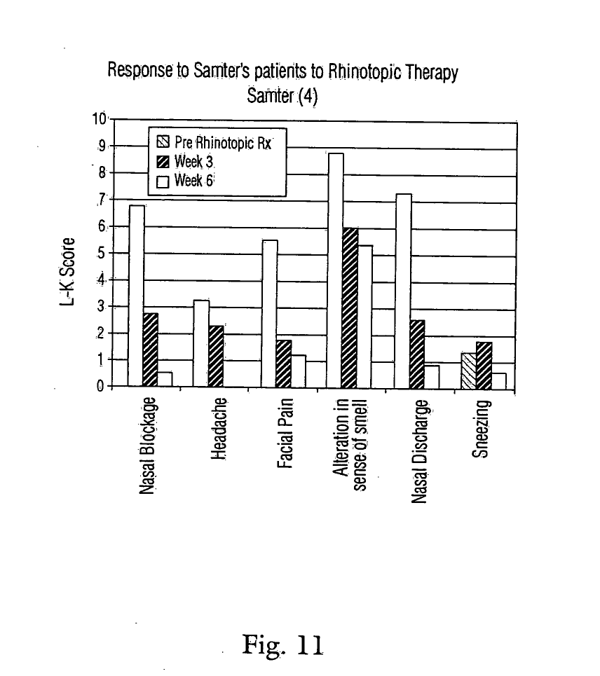

Presented herein in greater detail is a novel method and algorithm for the treatment of several complex sinus diseases for which standard medical/or surgical therapy is generally not effective, including: 1. MRSA (methicillin resistant Staphylococcus aureus) infection or colonization of the nasal cavity and nasal sinuses. MRSA is a stubborn organism that is often found in the nasal cavity and may remain as a source of bacterial dissemination. It is very difficult to eradicate using standard oral antibiotics, and the inventor has demonstrated that Rhinotopic Therapy is quite effective in clearing the organism from the nasal cavity (FIGS. 7 and 8). 2. Sinonasal sarcoidosis sinusitis a medical condition consisting of nasal obstruction, postnasal drainage, headache, and recurrent sinus infections due to sarcoid involvement of the sinuses. It is a very difficult disease to treat because it keeps recurring despite surgery and aggressive medical therapy (FIGS. 9 and 10). 3. Samter's triad a medical condition consisting of nasal/sinus polyps, asthma and aspirin sensitivity, which is also difficult to treat because the polyps keep recurring despite surgery and sustained medical therapy (FIG. 11). 4. Cystic fibrosis, a genetic multisystem disorder characterized by recurrent endobronchial infections, sinusitis, and pancreatic insufficiency with intestinal malabsorption. This disease is particularly difficult to treat and it keeps recurring despite surgery and aggressive medical therapy. These listed diseases are particularly amenable to Rhinotopic therapy.

Step 1: Topical Delivery of Antibiotics/Antimicrobials and Corticosteroids Through Sinus Nebulization.

Nebulization is an efficient and effective way to deliver drugs to the sinus cavities (10-18). It is superior to simple nasal irrigation/spraying in that it covers a wider deposition surface area and it does not rely on mucociliary clearance (which is usually impaired in CSR) to effect drug distribution. The optimal size for treating sinus infections through nebulization should be less than 5 μm (56). Hilton et al demonstrated in a cadaver study that particles in the 0.67-0.99 μm have improved efficiency of deposition in the sinuses after maxillary antrostomy, as compared to larger particles (57). The fundamental principals that determine efficiency of deposition of aerosolized particles in the paranasal sinuses are: The size of the sinus ostium, pressure/rate of flow of aerosol, and particle size (58). When attempting to identify the optimal sinus nebulizer, several factors should be taken in consideration, including practicality, ease of use of the device (in order to maximize patient compliance) and particle size to maximize drug delivery to the sinuses.

### Types of Nebulizers:

The inventor has found various available systems designed for delivery of aerosolized particles in the sinuses that differ in technology of aerosol delivery and aerosol particle size. These include the jet nebulizer, the ultrasonic nebulizer, and the vibrating mesh nebulizer, all of which are effective in this invention.

The jet nebulizers create an aerosol by forcing compressed air through a small “jet”, forcing the jetted air across the medication reservoir. The Venturi effect created adds the medication to the jet of air, which is further broken into smaller particles by collision with surfaces within the nebulizer. Compared with either ultrasonic or vibrating mesh nebulizer a smaller percentage of particles exiting a jet nebulizer are in the optimum under 5 μm most likely to reach the sinuses. The duration of treatment with jet nebulizers averages 15 minutes per 2.5 ml vials of medication, so compliance becomes a challenge when the patient is using more than one medication. The speed at which particles are expelled from the nosepiece of the jet nebulizer makes it necessary to wear eye protection and the size of the compressor and jet nebulizer together make this type of nebulizer less portable.

The ultrasonic nebulizers create a mist using ultrasonic sound waves to vibrate small particles off the top of a medication reservoir. The particle size in the aerosol is both smaller and more consistent than a jet nebulizer. The velocity of the mist leaving the nosepiece can be adjusted slow enough to prevent medication getting into the eyes. The disadvantage of ultrasonic nebulizers is that they are more complicated than other types of nebulizers, which creates compliance and teaching/training issues with use by patients in their homes.

The vibrating mesh nebulizer (VMN) is a small portable device that creates an aerosol mist by rapidly vibrating a mesh with hundreds of 4 to 8 μm holes, and allows a fast and uniform delivery of tiny aerosolized medication particles (average size 3.6 μm) inside the sinuses. The VMN do not require airflow from either a fan or compressor, which allows any aerosol created to remain in the nebulizer until airflow created by the patient's inhalation draws the aerosol into the patient's nostrils. Moreover, the volume of drug solution left in these devices when the nebulization has ceased is negligible, so there is potential to improve the cost-effectiveness of administering expensive medications. The additional advantage of the VMN units being silent, small, fast and portable made them very user friendly and the favorite nebulizer choice.

The inventor has chosen to nebulize antibiotics in addition to corticosteroids to target the infection and inflammation. It is believed that, infection and inflammation perpetuate each other in CRS and constitute the common pathways in refractory rhinosinusitis. The author believes that disrupting the biofilm three-dimensional structure, followed by actual removal of biofilm debris, is key in the treatment of refractory sinusitis. This can be done effectively through regular endoscopic mechanical cleansing of the sinuses by the treating rhinologist, followed with direct spraying of the topical antibiotic and corticosteroids preparations directly inside the sinus cavities using a mucosal atomizer device. In addition, the patients can practice twice daily saline hydrotherapy (which can be done through a saline sinus rinse device (including a pressure bottle, a Neti Pot, an electric irrigating device, and others). The inventor herein provides evidence that Rhinotopic therapy disrupts biofilms.

Step 3 Endoscopic Instillation of Antimicrobials+Corticosteroids Directly Inside the Sinus Cavities Using a Spray, Gel, Ointment, Lipospheres or Other Similar Vehicles.

The spray may be delivered using an atomizer device. The gel, ointment or lipospheres may be instilled in the sinuses using a cannula or a catheter under endoscopic guidance (or radiologic guidance). The gel (or ointment or lipospheres) can also be used for be used in conjunction with sinus surgery or in conjunction with balloon sinuplasty for CRS. The gel (or ointment) may consist of hydroxyl-ethylcellulose, ethyl Alcohol, propylene Glycol or other similar delivery vehicles. The gel (or ointment or lipospheres) may be delivered using a cannula under endoscopic visualization through a sinus endoscope, or also through a catheter in conjunction with balloon sinuplasty. Balloon Sinuplasty consists of using a catheter-based device to dilate blocked sinus windows, without tissue removal, similarly to catheter based coronary angioplasty that is used to dilate stenotic cardiac coronary arteries. Balloon sinuplasty is a surgical method that treats CRS, however, in some instances, the sinus mucosa is very inflamed or polypoid, and simply dilating the closed sinus opening with balloon sinuplasty is not enough to restore the health of the inflamed/infected sinus mucosa. In those cases, adding a topical gel that releases antimicrobials (antibiotics or antifungals) and/or corticosteroids helps restoring the sinus mucosa to health. The inventor provides evidence that rhinotopic therapy using a gel that contains antibiotics and corticosteroids is effective against refractory rhinosinusitis.

### Biofilm Disruption (Step 2+Step 4):

The inventor has demonstrated that Rhinotopic Therapy effectively removes mucosal biofilms. This is likely due to a combination of steps: these include 1. Reaching mucosal antibiotics levels above the MIC (Minimal Inhibitory Capacity), long enough to eradicate the biofilm bacteria. 2. Mechanical debridement and disruption of the biofilms' extracellular polymeric encasement, through weekly sinus suctioning/cleansing and daily hydrotherapy, with or without chelation using chemical chelating agents and surfactants that disrupt the bacterial cell membrane disruption. 3. Treating inflammation/polyps and granulation tissue using corticosteroids.

Preferred chelating agents that can be nebulized in the sinuses or placed topically in the gel include ethylenediamine-tetraacetic acid (EDTA), Citric Acid Zwitterionic Surfactant (CAZS), gallium nitrate, desferrioxamine, penicillamine, dimercaprol, etc.

Preferred surfactants include Polyethylene glycol 400; Sodium lauryl sulfate; sorbitan laurate, sorbitan palmitate, sorbitan stearate (available under the tradename SPAN® 20-40-60 etc.); polysorbates including, but not limited to, polyoxyethylene (20) sorbitan monolaurate, polyoxyethylene (20) sorbitan monopalmitate, polyoxyethylene (20) sorbitan monostearate (available under the tradename TWEEN® 20-40-60 etc.); and Benzalkonium chloride.

Preferred mucolytic agents (Step 4) are Acetylcysteine and Dornase Alpha, etc. Preferred anti-infective agents include Penicillins, Cephalosporins, Macrolides, Sulfonamides, Quinolones, Aminoglycosides, BetaLactam antibiotics, Linezolid, Vancomycin, Amphotericin B, and Azole antifungals.

Step 4, Hydrotherapy:

Hydrotherapy is an effective means of mechanically disrupting biofilms attachment and removing them from open wounds in the orthopedic literature. Saline delivered by a hydrodynamic force is successful in significantly reducing biofilms cell count (51, 52, 53) and was also effective to humidify dry sinus mucosa and eliminate foreign bodies (such as crusts) that could damage the mucosa and provide excellent substrates for bacterial attachment and biofilms formation (29). Nasal saline irrigation also mechanically rinses aeroallergens like pollen, mold, dust, and other pollution particulates that affect mucociliary flow rates. Effective in this invention, hydrotherapy can be delivered through saline bottle, squirt bottle or through an irrigating mechanical device.

In order to assess the biofilm response to treatment, Colony Forming Units (CFU) assay was used. This is the most reliable quantifiable biofilm assay that is available to date. Sinus mucosal biopsy issue is homogenized and bacterial biofilms are grown on fresh agar plates and then counted, allowing a quantitative assessment of the biofilm response to Rhinotopic therapy. We have found that Rhinotopic Therapy causes 90% elimination &/or reduction in biofilm bacterial CFU (Colony Forming Units) counts.

### Effect of Rhinotopic Therapy on Biofilm Bacterial CFU (Colony Forming Units) Counts

| Pre-Rhinotopic Bacteria | Pre-Rhinotopic Rx (CFUs) | Post-Rhinotopic Rx (CFUs) |
|---|---|---|
| Respiratory Flora | CFUs: 540 cells/ml, | No Growth |
| | 3 diff bacterial morphologies; all | |
| | gram positive cocci | |
| MRSA | CFUs: 100x cells/ml; gram | No Growth |
| | positive cocci in clusters | |
| | (staphylococci). | |
| Pantoea agglomerans | Done CFUs: 90,000,000 cells/ml; | 50 cells/ml (Gram + cocci; |
| | Pseudomonas. | staphylococci) |
| E. coli + | Total CFUs: 4500 cells/ml-: | No Growth |
| Pseudomonas aeroginosa | 1. CFUs: 6.5 × 102; Pseudomonas | |
| | 2. CFUs: 3.5 × 103 staphylococci | |
| Serratia Marscecens | CFUs: 2250 cell/ml; | No Growth |
| | small gram negative rods | |
| | (morphology consistent with | |
| | Haemophilus). | |
| Staph aureus | CFUs: 150 cells/ml | 460 staph and 100 Pseudomonas |
| | | Aeroginosa |
| Staph Aureus + Pseudomonas | | No Growth |
| Aeroginosa | | |
| MRSA + | CFUs: 250 cells/ml; gram positive | No Growth |
| Staph coagulase negative | cocci in clusters | |
| | (staphylococci). 2ND ONE DONE | |
| | WAS NEGATIVE | |
| MRSA | 2,120,000 cells/ml gram negative | No Growth |
| | rods | |
| MRSA | 6,400 cell/ml | No Growth |

Complex sinus diseases for which standard medical/or surgical therapy is usually not effective:
a. MRSA (methicillin resistant Staphylococcus aureus) FIGS. 7 and 8
b. Sinonasal sarcoidosis FIG. 9, 10
c. Samter's triad FIG. 11
d. Cystic fibrosis is a genetic multisystem disorder characterized by recurrent endobronchial infections, sinusitis, and pancreatic insufficiency with intestinal malabsorption. This disease is particularly difficult to treat and it keeps recurring despite surgery and aggressive medical therapy.
a. MRSA (Methicillin Resistant Staphylococcus Aureus): Staphylococcus aureus (S. aureus) is a bacterium that often causes infection or colonization of the nose and paranasal sinuses. The difference between colonization and infection is that infection causes symptoms but the patient does not symptoms when colonized. Infection symptoms included nasal discharge, congestion, headache, systemic manifestation of disease such as fever, malaise. 70% to 90% of all individuals are intermittently colonized with S. aureus (methicillin susceptible or resistant) in the anterior nares. S. aureus permanently colonized the anterior nares of about 20% to 30% of the general population. S. aureus that is resistant to methicillin or oxacillin is reported as MRSA. Hospital workers are more likely to be colonized than persons in the general population, presumably because of increased exposure, and they are a potential hazard to spread MRSA to their healthy patients. MRSA is a stubborn organism that is very difficult to eradicate using standard oral antibiotics and that usually require prolonged intravenous antibiotics, such as Vancomycin, however this carries significant side effects that include toxicity to the kidneys and the inner ear. The standard protocol for MRSA carrier nasal includes a topical nasal application of mupirocin antibiotic ointment; however the treatment is successful in 50% to 60% of the cases at best. We have shown that Rhinotopic therapy using nebulized (+/−) topical gel mupirocin or vancomycin is effective in treating MRSA sinusitis and controlling symptoms between 75 to 90% of the patients. FIGS. 7 and 8.
b. Sinonasal Sarcoidosis: Sarcoidosis that involves the sinuses presents with nasal obstruction, postnasal drainage, headache, and recurrent sinus infections due to sarcoid involvement of the sinuses. It is difficult to treat because the disease keeps recurring despite surgery and aggressive medical therapy. The treatment consists usually of large doses of prolonged oral corticosteroids (which have significant side effects) and frequent sinus surgery procedures, however these result at best offer a temporary relief of the symptoms. We have shown that Rhinotopic therapy can be a very effective adjuvant treatment for sinonasal sarcoidosis. FIGS. 9 and 10
c. Samter's triad is a medical condition consisting of nasal/sinus polyposis (+/−rhinosinusitis), asthma and aspirin sensitivity. It is difficult to treat because the polyps keep recurring despite surgery and aggressive medical therapy. We have shown that Rhinotopic therapy is effective in treating Samter's triad. FIG. 11
d. Cystic fibrosis is a genetic multisystem disorder characterized by recurrent endobronchial infections, sinusitis, and pancreatic insufficiency with intestinal malabsorption. The sinusitis in this disease is particularly difficult to treat and it keeps recurring despite surgery and aggressive medical therapy. Rhinotopic therapy reaches mucosa! levels of antibiotics that are above the MIC levels needed to kill the local organisms.

### Clinical Treatment

1. “Rhinotopic therapy”: This new treatment method, called “Rhinotopic therapy” involves topical antibiotics and corticosteroids+topical debridement and hydrotherapy. It offers a medical alternative to patients with CRS that which has been refractory to conventional medical and surgical treatment. Rhinotopic therapy in detail:
   Step 1: Regular topical delivery of antibiotics/antimicrobials and corticosteroids to the infected/inflamed sinuses using a nebulizer, or atomizer by the patient twice daily generally for from six to eight weeks.
   Step 2: Endoscopic debridement and cleansing of the sinus cavities from debris and from biofilms that adhere to the sinus mucosa is performed by the physician or skilled technician once a week.
   Step 3: Endoscopic instillation (by spray, gel/ointment, polymer or lipospere) of antibiotics/antimicrobials and corticosteroids directly inside the sinus cavities or the areas around the sinuses, such as the middle meatus or the inferior meatus. This treatment is performed by the physician or skilled technician once a week and preferably along with Step 2.
   Step 4: Regular hydrotherapy with saline douches or irrigations (with or without chelating agents or surfactants or mucolytics) that inhibit bacterial/fungal attachment to the sinus mucosa, performed by the patient twice daily.

These above treatment steps are carried out for about six to eight weeks, with about six weeks being optimal. A sustained therapy for more than 8 weeks may however be indicated in resistant infections.

While specific numeric amounts of medicaments have been set forth in the above treatment protocol, it is to be understood that those skilled in the art could vary these amounts without departing from the spirit of this invention.

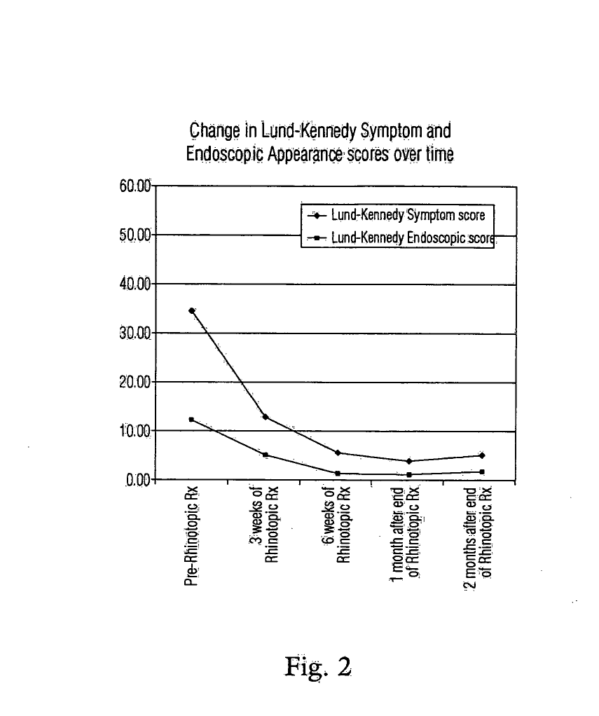

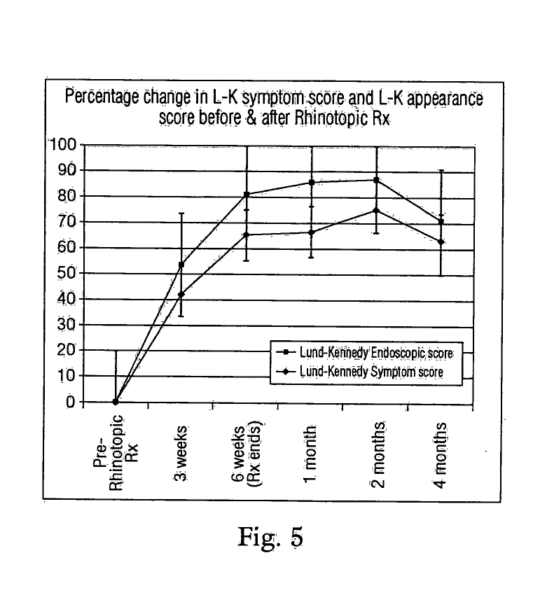

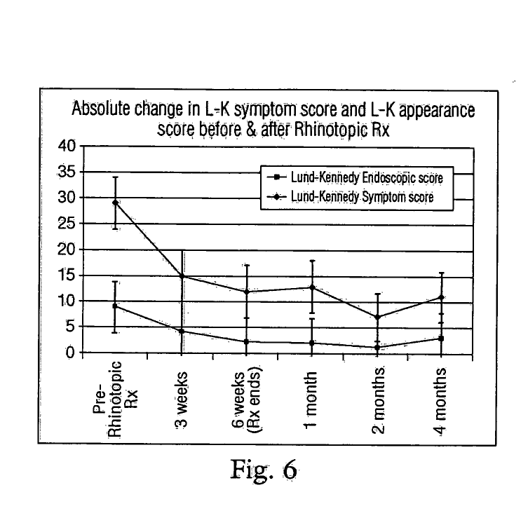

The inventor has successfully treated 24 patients with refractory sinusitis with topically aerosolized corticosteroids and antibiotics followed by weekly application of a spray of the corticosteroids and antibiotics using a mucosa! atomizing device (FIGS. 2, 3 and 4) and also successfully treated 30 patients with refractory sinusitis with topically aerosolized corticosteroids and antibiotics followed by weekly application of a gel containing corticosteroids and antibiotics using a cannula. (FIGS. 5 and 6). Nebulizer-treatment was self-administered twice daily by the patients for a period of 6 weeks, using the small particle vibrating mesh nebulizer (VMN) (Aerogen LTD. Galway, Ireland) which creates an aerosol mist by rapidly vibrating a mesh with hundreds of 4 to 8 μm holes, and allows a fast and uniform delivery of tiny aerosolized medication particles to the sinuses. The antibiotics were chosen selected on the basis of culture-specific sensitivity. In addition, patients practiced hydrotherapy with sinus rinses using nasal saline twice daily. In addition, endoscopic sinus cleansing and biofilm removal were performed weekly by the treating rhinologist, followed in one group with spraying of the topical antimicrobials (antibiotics or antifungals) and corticosteroids preparations directly inside the sinus cavities using a mucosa atomizer (20 patients), and in another group with direct instillation of a gel that contains antimicrobials (antibiotics or antifungals) and corticosteroids (30 patients).

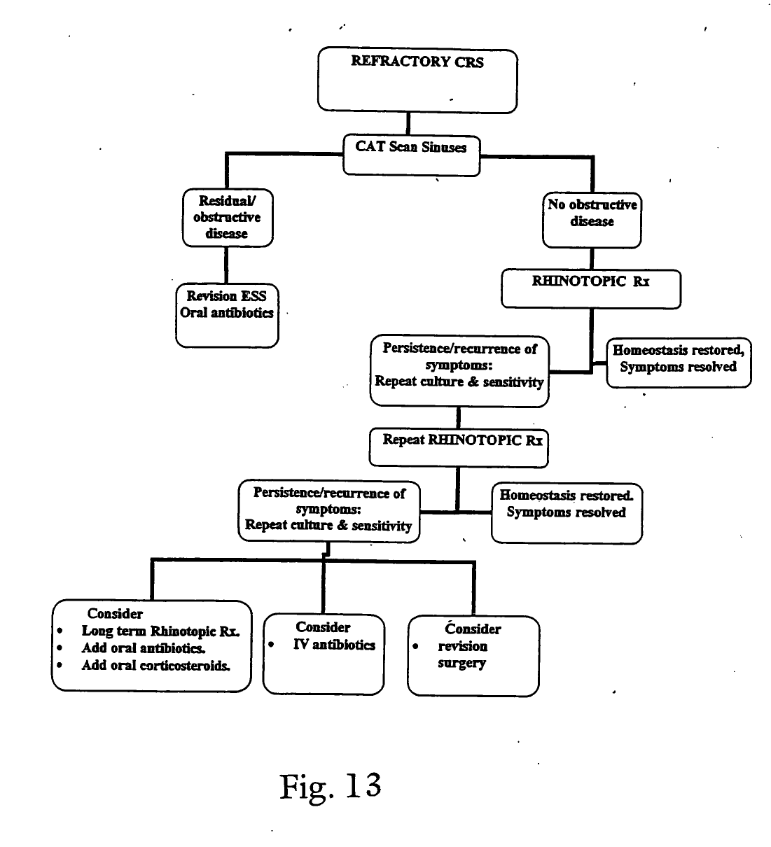

With reference to the flowchart (algorithm) FIG. 13 the method for treating refractory CRS (chronic rhinosinusitis) is herein described in narrative form as follows:

A patient with refractory chronic rhinosinusitis is first subjected to a CAT Scan of the sinuses.

If residual obstructive disease is found, the patient is subjected to revision ESS (endoscopic sinus surgery) and oral antibiotics; and if no obstructive disease is found, continue with Rhinotopic Rx (Rhinotopic Therapy).

If Homeostasis is restored and symptoms are resolved, discontinue treatment; if there is persistence/recurrence of symptoms: repeat culture and sensitivity and repeat Rhinotopic Rx (Rhinotopic Therapy).

If with the repetition of Rhinotopic Rx, homeostasis is restored and symptoms resolved, discontinue treatment, and if after repeating Rhinotopic Rx there is persistence/recurrence of symptoms: repeat culture and sensitivity and consider the following treatments:

Treatment 1: Long term Rhinotopic Rx
Add oral antibiotics
Add oral corticosteroids
   Treatment 2: IV antibiotics

### Treatment 3: Revision

Surgery
   to relieve the symptoms of refractory chronic rhinosinusitis.

### Table and Clinical Studies

### Part I

The first part is a prospective study that includes 20 patients with refractory CRS, treated in with Rhinotopic therapy spray at a tertiary rhinology fellowship-training program. The patients were placed on the rhinotopic protocol only after they failed extended standard medical therapy. The latter is defined, for the purpose of this study, as three or more repeated courses of different oral antibiotics along with nasal corticosteroids sprays and nasal saline rinses (each lasting at least 3 weeks), within a 6 month-period, along with oral corticosteroids when indicated (in case of significant polyposis or Samter's triad). Patients who failed extended medical therapy were given the option of revision sinus surgery or rhinotopic therapy; all 20 patients chose rhinotopic therapy and were consented accordingly. All patients were over 18 years of age, had one or more prior endoscopic sinus surgery, signs and symptoms of recurrent or persistent CRS, along with endoscopic and CT evidence of patient sinus ostia with absence of obstructive disease. The patients were subjected to a battery of investigational tests upon enrollment, including CT scan of the sinus, allergy workup (RAST test) along with swab aerobic and anaerobic cultures taken from the ethmoid/maxillary sinus cavities under endoscopic guidance. They were treated over a period of 6 weeks with a topically aerolized corticosteroid along with an antibiotic that was chosen based on culture and sensitivity (Table I). The aerosols were self-administered twice daily using a small particle vibrating mesh nebulizer (VMN, Aerogen LTD, Galway, Ireland). The VMN creates an aerosol mist by rapidly vibrating a mesh with hundreds of 4 to 8 μm holes, and allows a fast and uniform delivery of tiny medication particles to the sinuses. The aerolized corticosteroid used in this study was mometasone furoate, with a concentration of 0.6 mg per 2 ml of 0.9% sodium chloride base, adjusted with 10% w/v HCl to a pH range of 4.4 to 4.8. The suspension contained a small amount of surfactant, polysorbate 80. The aerosols preparations were provided by ASL Pharmacy (Camarillo, Calif.) and were used sequentially, starting with the mometasone and then followed by the antibiotic. The patients were instructed to practice hydrotherapy, consisting of twice daily nasal saline rinses using the NeilMed® Sinus Rinse (Santa Rosa, Calif.), to be followed by self-administration of the nebulized treatment. No oral antibiotics were given throughout the duration of the study or the follow up period. The patients were examined in the clinic weekly for a period of 6 weeks, at which time a thorough endoscopic debridement of the sinus crusts and mucous was performed, followed by aerosolization of the same antibiotic and corticosteroid directly in the sinus cavities, using a mucosal atomizing device. One month after completion of treatment, a repeat swab culture was taken from the ethmoid/maxillary sinus cavities under endoscopic guidance; all patients were followed up one month, two months and six months (or longer) following the completion of therapy.

CT scans were reviewed prior to treatment and evaluated following the Lund-Mackay radiologic scoring system (9). Clinical response to treatment was monitored using the Lund-Kennedy (LK) symptoms score and the LK endoscopic appearance score (9). This was done before initiation of rhinotopic therapy, at treatment weeks 3 and 6, and also one month, two months and six months after the completion of treatment. The LK symptoms score evaluated nasal congestion and obstruction, headache, facial pain, alteration I the sense of smell, nasal discharge, and sneezing. The LK endoscopic appearance score evaluated the presence or absence of nasal polyps, edema, discharge, scarring, and crusting on each side of the nasal cavity. Statistical analysis was done using Student's T-test.

### Results

The mean age of the remaining 20 patients was 48 years (range: 13-76) years). There were 12 women and 8 men. All patients had prior endoscopic sinus surgery, several at outside institutions (range: 1 to 8 surgeries), consisting at a minimum of one complete ESS, including bilateral spheno-ethmoidectomy, maxillary antrostomy and frontal exploration. Two patients had Samter's triad. All patients had received and failed at least three courses of oral antibiotics and nasal corticosteroids during the 6 months prior to inclusion in the study. Eight patients were also treated with oral corticosteroids (those patients with significant polyposis or Samter's triad). One patient elected to try initially intravenous ceftazidine; she improved transiently but her symptoms recurred and she was later placed on the rhinotopic protocol. Review of the CAT scans and endoscopic examination before starting treatment confirmed the absence of sinus obstruction or significant residual ehtmoid disease. A few patients had some degree of narrowing of the frontal sinus and/or the spheno-ehtmoidal recess by polyps or polypoid mucosa. The patients' demographics, including the organisms cultured and the type of nebulized treatment are presented in Table I. The most commonly cultured bacteria were Staphylococcus aureus (n=9, 45%) including 2 MRSA and Pseudomonas aeruginosa

(n=5, 25%). All the recovered organisms were resistant to oral antibiotics. Anaerobic cultures were obtained but no anaerobic bacteria were retrieved. The nebulized antibiotics included tobramycin (16 patients) and vancomycin (3 patients). One patient grew Aspergillus and was treated with amphotericin B. The mean follow up period after completion of treatment was 33 weeks. Seventeen patients cleared after a single rhinotopic course and remained symptom-free during the follow up period. Three patients had gradual recurrence of their symptoms during the initial 6 months follow up period and were successfully treated with rhinotopic therapy. Two Samter's patients relapsed beyond the 6 months of treatment. One patient developed left frontal sinus obstruction that necessitated frontal exploration.

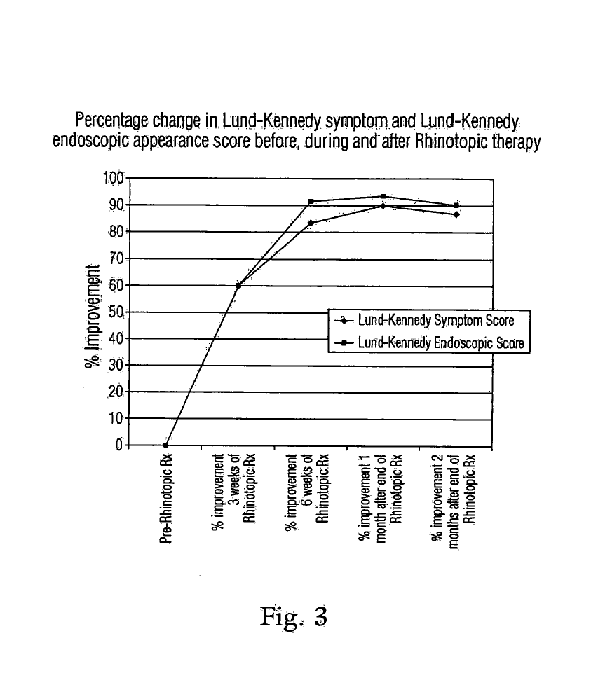

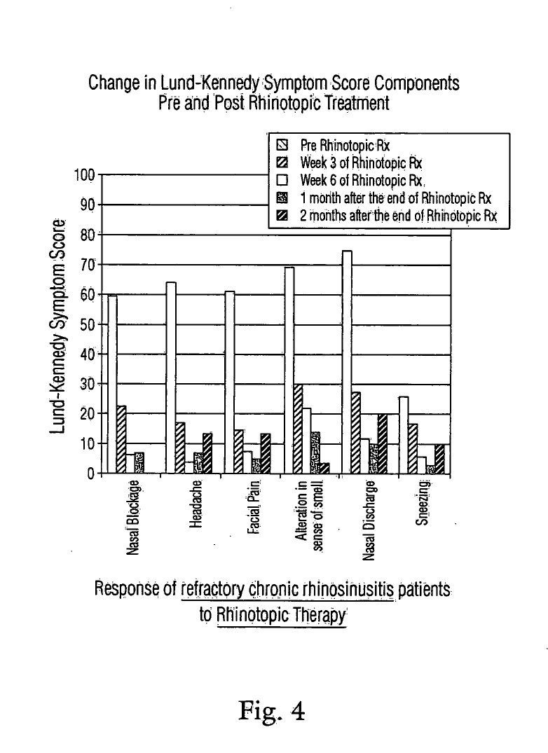

The mean LK symptom and LK endoscopic appearance scores showed a statistically significant improvement with treatment, with P value <09991 throughout the treatment period and up to 6 months after treatment completion (FIG. 2). The percentage improvement in the mean LK scores during and after treatment is shown in FIG. 3. Each component of the LK symptom score was assessed separately and plotted (FIG. 4). The most improvement was noted for headache, facial pain and nasal obstruction, the least for the alteration in the sense of smell. Repeated swab culture conducted one month after completion of treatment revealed no growth in 65% of the cases, normal respiratory flora in 25%, and persistence (or recurrence) of pathogenic organisms in 10%.

There were a few mild side effects: two patients complained of mild cough and throat irritation and one patient had mild transient epistaxis while on the nebulizer, but this did not prevent them from finishing the 6-week treatment. One patient developed an allergic reaction to vancomycin (skin rash over the upper lip), elected to stop the treatment and dropped out.

TABLE I
Patients Demographics.

| | Age/ | | | Number of | Posttreatment |
|---|---|---|---|---|---|
| Initials | Sex | Pretreatment Organisms | Nebulized Drugs | Treatments | Organisms |
| NS | 76/F | Pseudomonas | Levaquin and | 3 | Pseudomonas |
| | | aeruginosa | Mometasone | | aeruginosa |
| MB | 50/F | Methicillin resistant | Vancomycin and | 2 | Normal |
| | | Staphylococcus aureus | Mometasone | | respiratory flora |
| EC | 52/F | Pseudomonas | Levaquin and | 1 | No growth |
| | | aeruginosa | Mometasone | | |
| AD | 48/M | Staphylococcus | Tobramycin and | 1 | No growth |
| | | marcescens | Mometasone | | |
| LL | 51/F | Staphylococcus aureus/ | Tobramycin and | 1 | No growth |
| | | Pseudomonas | Mometasone | | |
| | | aeruginosa | | | |
| KJ | 45/M | Staphylococcus aureus | Tobramycin and | 1 | No growth |
| | | | Mometasone | | |
| RP | 46/M | Staphylococcus aureus | Tobramycin and | 1 | No growth |
| | | | Mometasone | | |
| GD | 56/F | Staphylococcus aureus | Tobramycin and | 1 | Normal |
| | | | Mometasone | | respiratory flora |
| SR | 31/M | Staphylococcus aureus | Tobramycin and | 1 | No growth |
| | | | Mometasone | | |
| JB | 56/F | Pseudomonas | Tobramycin and | 1 | Normal |
| | | aeruginosa | Mometasone | | respiratory flora |
| NTC | 32/F | Aspergillus fumigatus | Amphotericine and | 1 | Staphylococcus |
| | | | Mometasone | | aureus |
| PA | 57/F | Staphylococcus aureus | Tobramycin and | 1 | No growth |
| | | | Mometasone | | |
| GJ | 53/M | Methicillin resistant | Vancomycin and | 1 | Normal |
| | | Staphylococcus aureus | Mometasone | | respiratory flora |
| TSS | 44/F | Citrobacter Koseri | Tobramycin and | 1 | No growth |
| | | | Mometasone | | |
| JS | 31/M | Pseudomonas | Tobramycin and | 1 | No growth |
| | | aeruginosa | Mometasone | | |
| CH | 59/F | Staphylococcus aureus | Tobramycin and | 2 | No growth |
| | | | Mometasone | | |
| BD | 67/F | Eikenella corrodens | Tobramycin and | 1 | Normal |
| | | | Mometasone | | respiratory flora |
| CL | 50/M | Staphylococcus | Tobramycin and | 1 | No growth |
| | | marcescens | Mometasone | | |
| MA | 62/M | Pseudomonas | Levaquin and | 1 | No growth |
| | | aeruginosa | Mometasone | | |
| DT | 48/F | Staphylococcus aureus | Levaquin and | 1 | No growth |
| | | | Mometasone | | |
| MC | 45/F | Staphylococcus | Tobramycin and | 1 | No growth |
| | | intermedius | Mometasone | | |
| DB | 52/M | Klebsiella pneumoniae | Tobramycin and | 1 | No growth |
| | | | Mometasone | | |
| LM | 42/F | Staphylococcus aureus | Tobramycin and | 1 | No growth |
| | | | Mometasone | | |

### Part II:

This second part is a prospective study that includes 30 patients with refractory CRS, treated with Rhinotopic therapy gel that contains antibiotics and corticosteroids. The mean LK symptom and LK endoscopic appearance scores showed a statistically significant improvement with treatment, with P value <0.0001 throughout the treatment period and at 1 month and 2 months after treatment completion (FIG. 5). The percentage improvement in the mean LK scores during and after treatment is shown in FIG. 6.

### Kit Feature

A special feature of the herein disclosed invention is a kit 10 shown in FIG. 12 containing components necessary for performing Rhinotopic Therapy. The kit 10 would contain for performing Step 1 a mucosal atomizing device attached to a syringe loaded with the appropriate medication 12 and containers of an antibiotic 14 and corticosteroid 16. Step 3 there are mucosal atomizing devices or cannulas to deliver the antibiotic/corticosteroids spray or gel for instilling antimicrobials 14 and corticosteroids 16; and for Step 4 a saline powder packets 18 to reconstitute with water into a douche, with or without added components is shown. Also shown as part of FIG. 12 is a kit 20 containing two layers (not shown) of antimicrobials 22 and corticosteroids 24 for use by the physician in Step 3.

Obviously, many modifications may be made without departing from the basic spirit of the present invention. Accordingly, it will be appreciated by those skilled in the art that within the scope of the appended claims, the invention may be practiced other than has been specifically described herein.

1. In the treatment of the disease of rhinosinusitis, wherein the sinus lining is inflamed due to bacterial, viral and/or microbial infection causing structural issues such as sinus ostium blockage; wherein the conventional medical therapy consists of antibiotics and/or topical nasal corticosteroids, which is not effective in an appreciable segment of the patients and may have to be repeated; and wherein, if the patient does not respond to the aforesaid medical therapy, sinus surgery may be recommended and, if performed, is not always effective and may have to be repeated; a multi-faceted rhinotopic therapy comprising the steps of nebulizing small-particle antimicrobials/antibiotics and/or corticosteroids for the treatment of the infection and/or inflammation, respectively; cleaning the sinus cavities from debris and/or biofilms sticking to the sinus mucosa, applying antimicrobials and/or corticosteroids directly inside the sinus cavities and/or the areas around the sinuses; and including the step of regular hydrotherapy, thereby avoiding repeated medical therapy and/or sinus surgery, and thereby providing a highly effective treatment modality for rhinosinusitis without significant side effects.

2. A method for treating refractory or recalcitrant rhinosinusitis comprising the steps of:
(1) applying to the inflamed and infected sinuses using a nebulizer, a topical application of antimicrobials/antibiotics and corticosteroids,
(2) endoscopic debridement and cleansing of the sinus cavities from debris and from biofilms that adhere to the sinus mucosa,
(3) endoscopic instillation of antimicrobials/antibiotics plus corticosteroids directly inside the sinus cavities or the areas around the sinuses, and
(4) hydrotherapy with saline douches or irrigations that disrupt/inhibit bacterial/fungal biofilms and their attachment to the sinus mucosa, to bring about a highly effective treatment for refractory or recalcitrant rhinosinusitis.

3. The method of claim 2, wherein the antimicrobials/antibiotics plus the corticosteroids of step (1) are endoscopically instilled around the middle meatus or inferior meatus of the sinuses, and the hydrotherapy of step (4) is effected using saline douches or irrigations with chelating agents, surfactants or mucolytics.

4. The method of claim 3, wherein the endoscopic instillation of antimicrobials/antibiotics plus corticosteroids of step (3) is done through a topical atomizing device or by applying a gel, ointment, polymer or lipospheres that contain antimicrobials/antibiotics plus corticosteroids and releases them locally as the composition degrades.

5. In the method of claim 2, wherein in step (2) nebulized antibiotics are employed for the treatment of bacterial biofilms along with nebulized corticosteroids for the treatment of inflammation/granulation; and with biofilm debridement and cleansing followed by endoscopic instillation of small and/or large antimicrobials/antibiotics and corticosteroids into the sinus cavities.

6. In the method of claim 5, wherein the small particles are nebulized employing a vibrating mesh nebulizer, or larger particles are nebulized using a jet nebulizer or an ultrasonic nebulizer.

7. In the method of claim 2, wherein the antimicrobials/antibiotics and/or corticosteroids/anti-inflammatories are delivered employing a gel or ointment comprising hydroxethylcellulose or propylene glycol.

8. In the method of claim 7, wherein the gel or ointment is delivered using a cannula under endoscopic visualization or by way of a catheter under fluoroscopy guidance, transillumination guidance or computerized tomography guidance.

9. The method of claim 8, wherein the gel is administered to the sinuses employing balloon sinuplasty which employs a small flexible balloon catheter to open blocked sinus passageways and at the same time restoring normal drainage and ventilation of the sinus.

10. The method of claim 2, wherein the steps of the process are carried to treat a disease selected from the group consisting of methicillin resistant Staphylococcus aureus (MRSA), sinonasal sarcoidosis, Samter's triad/polyposis and cystic fibrosis.

11. The method of claim 10, wherein methicillin resistant Staphylococcus aureus (MRSA) is treated using nebulized topical gel mupirocin or vancomycin.

12. (canceled)

13. A kit of components for carrying out the method of claim 1 for refractory or recalcitrant rhinosinusitis comprising antimicrobials/antibiotics and corticosteroids solutions and an atomizer delivery device for the sinuses.

14. A kit of components for carrying out the method of claim 1 for refractory or recalcitrant rhinosinusitis comprising antimicrobials/antibiotics and corticosteroids gels/ointments and a cannula delivery device for the sinuses.

15. A kit of components for carrying out the method of claim 2 for refractory or recalcitrant rhinosinusitis comprising antimicrobials/antibiotics and corticosteroids solutions and an atomizer delivery device to the sinuses for use in Step 3 along with a nebulizer for carrying out Step 1, and an atomizing delivery device and douche packets for use in Step 4.

16. A kit of components for carrying out the method of claim 2 for refractory or recalcitrant rhinosinusitis comprising antimicrobials/antibiotics and corticosteroids gel/ointments and cannula delivery devices to the sinuses for use in Step 3 along with a nebulizer for carrying out Step 1, an atomizing delivery device and a douche for use in Step 4.

17. The method of claim 2 wherein a kit containing a nebulizer and a six to eight week supply of antibiotics, corticosteroids and saline douche packets is supplied to carry out the method of treating refractory or recalcitrant rhinosinusitis.

18. A method for treating refractory chronic rhinosinusitis in a patient comprising the steps of:
first subjecting said patent to a CAT Scan of the sinuses and if residual obstructive disease is found, the patent is subjected to revision endoscopic sinus surgery and oral antibiotics;
if no obstructive disease is found, continue with rhinotopic therapy;
if homeostasis is restored and symptoms are resolved, discontinue treatment;
if there is persistence/recurrence of symptoms:
repeat culture and sensitivity and repeat rhinotopic therapy;
if with the repetition of rhinotopic therapy, homeostasis is restored and symptoms resolved, discontinue treatment, and if after repeating rhinotopic therapy there is persistence/recurrence of symptoms: repeat culture and sensitivity and consider the following treatments:
Treatment 1—long term rhinotopic therapy; add oral antibiotics; add oral corticosteroids;
Treatment 2—IV antibiotics;
Treatment 3—Revision surgery;
to relieve the symptoms of refractory chronic rhinosinusitis.
